(19) United States
(12) Patent Application Publication
(10) Pub. No.: US 2020/0332367 A1
(43) Pub. Date: Oct. 22, 2020

(54) MSI From Liquid Biopsies

(71) Applicant: Nantomics, Culver City, CA (US)

(72) Inventor: Xu Huang, Culver City, CA (US)

(73) Assignee: Nantomics, Culver City, CA (US)

(21) Appl. No.: 16/754,102

(22) PCT Filed: Oct. 18, 2018
(86) PCT No.: PCT/US2018/056557

§ 371 Date: Apr. 6, 2020

Related U.S. Application Data
(60) Provisional application No. 62/574,718, filed on Oct. 19, 2017.

(51) Int. Cl. C12Q 1/6886; C12Q 1/686

(52) U.S. Cl. CPC C12Q 1/6886; C12Q 1/686; C12Q 2565/137; C12Q 2565/125; C12Q 2600/156

(57) ABSTRACT

Methods for detection of MSI in a solid tumor without the need of tumor tissue are presented. In especially preferred methods, a blood sample from a patient is used to isolate ctDNA from serum and nuclear DNA from leukocytes. So obtained DNA is then employed as source material for MSI detection, typically via amplification of one or more MSI loci. In especially preferred aspects, size analysis of amplicons is performed without the need for fluorescent markers.

This application claims priority to our copending U.S. Provisional Patent application with the Ser. No. 62/574,718, which was filed Oct. 19, 2017, incorporated by reference herein.

### FIELD OF THE INVENTION

The field of the invention is profiling of omics data as they relate to cancer, especially as it relates to the identification of microsatellite instability (MSI) in solid tumor cells from blood and other biological fluids.

### BACKGROUND OF THE INVENTION

The background description includes information that may be useful in understanding the present invention. It is not an admission that any of the information provided herein is prior art or relevant to the presently claimed invention, or that any publication specifically or implicitly referenced is prior art.

All publications and patent applications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Microsatellites are typically short, tandem repeated DNA sequences with 1-6 base pairs in length. These repeats are distributed throughout the genome and often vary in length from one individual to another, due to differences in the number of tandem repeats at each locus. More recently, microsatellite markers have been used to detect MSI (microsatellite instability), which is a form of genomic instability. MSI is characterized as a change in length of a microsatellite allele due to insertion or deletion of repeat units during DNA replication and failure of the DNA mismatch repair system to correct these errors.

Typically, MSI analysis involves comparing allelic profiles of microsatellite markers generated by amplification of DNA from matching normal and test samples, which may be mismatch-repair (MMR) deficient. Alleles that are present in the test sample but not in corresponding normal samples indicate MSI. Commonly, mononucleotide repeat markers included in MSI analysis are selected for high sensitivity and specificity to alterations in samples containing mismatch repair defects, and most preferably such mononucleotide repeat markers are quasi-monomorphic (i.e., almost all individuals (e.g., at least 90%, more typically at least 95% of individuals) are homozygous for the same common allele for a given marker). As will be readily appreciated, use of quasi-monomorphic or monomorphic markers simplifies data interpretation, and there are numerous suitable loci known in the art to identify MSI. For example, suitable loci are described in U.S. Pat. Nos. 6,150,100, 7,662,595, US2003/0113723, US 2015/0337388, and WO 2017/112738.

There are also numerous methods known in the art to detects MSI from selected loci and most typically include PCR based methods. For example, a commercially available test kit for MSI analysis is offered by Promega Corporation (2800 Woods Hollow Road, Madison, Wis. 53711-5399 USA). Alternatively, MSI may also be inferred from a specific omics analysis as is described in US 2017/0032082.

Most samples for MSI analysis are fresh tissue samples from surgery or from biopsies, or formalin-fixed, paraffin-embedded (FFPE) samples. However, obtaining sufficient high-quality DNA from FFPE samples can be problematic since DNA is often degraded due to prolonged or improper fixation of the tissue sample before embedding in paraffin. Yet other attempts to detect MSI were made by correlating overall cfDNA quantities in blood with MSI as described in In Vivo 28: 349-354 (2014), but no correlation was found in this study between MMR proficient and MMR deficient samples. Thus, even though various systems and methods are known to determine MSI, all or almost all of them suffer from one or more disadvantages. Most typically, samples with high purity and stability can only be obtained using invasive procedures or surgery, while FFPE samples often suffer from lack of purity and/or stability.

Thus, there remains a need for improved methods of analyzing MSI in cancer, especially where biological samples can be obtained in a simple and safe manner.

### SUMMARY OF THE INVENTION

The inventive subject matter is directed to various methods of detection of MSI from a patient sample that is not a tumor sample. Most advantageously, MSI can be detected from a single whole blood sample that provides ctDNA from serum and nuclear DNA from cells in the blood (most typically leukocytes). The ctDNA and the nuclear DNA are preferably used as starting material for amplification and size determination as samples for tumor and matched normal, respectively.

In one aspect of the inventive subject matter, the inventors a contemplate a method of detecting microsatellite instability (MSI) in a solid tumor that includes a step of isolating a cell-containing fraction (preferably buffy coat fraction) and a cell-depleted fraction from a blood sample of a patient having the solid tumor, and another step of isolating cell-free circulating tumor DNA (ctDNA) from the cell-depleted fraction. In a further step, nuclear DNA is isolated from the cell-containing fraction, and at least one MSI locus is amplified in the ctDNA and in the nuclear DNA. A size difference is then detected between the amplified MSI locus in the ctDNA and the amplified MSI locus in the nuclear DNA.

Most typically, the step of amplifying at least one MSI locus comprises amplifying at least three MSI loci, or at least five MSI loci. It is further contemplated that at least one MSI locus is a quasi-monomorphic or monomorphic repeat marker, and/or that at least one MSI locus includes a mononucleotide repeat or a dinucleotide repeat. For example, suitable MSI loci include NR-21, BAT-26, BAT-25, NR-24, and MONO-27. In further contemplated aspects, preferred steps of detecting the size difference is done by capillary electrophoresis, polyacrylamide gel electrophoresis, mass spectroscopy, chip-based microfluidic electrophoresis (Methods Mol Biol. 2013; 919:287-96), or denaturing high performance liquid chromatography. Additionally, it is contemplated that the step of detecting the size difference comprises a step of comparing peak shape and position in an elution profile of a chromatogram of the amplified MSI locus. For example, the peak shape is area under the curve and/or peak height, or that the step of comparing comprises a step of independent component analysis.

In another aspect of the inventive subject matter, the inventors contemplate a method of detecting microsatellite instability (MSI) in a solid tumor that includes a step of obtaining tumor and matched normal DNA from a blood sample of a patient having the solid tumor, and a further step of using the tumor and matched normal DNA from the blood sample as source material for MSI analysis.

Preferably, the tumor DNA is ctDNA, and/or the matched normal DNA is DNA from leukocytes. Moreover, it is contemplated that the MSI analysis includes a step of PCR amplification of at least one MSI locus, and/or that the MSI analysis includes a step of capillary electrophoresis and fluorescence detection. Alternatively, the MSI analysis may include a step of size separation chromatography without fluorescence detection.

Viewed from a different perspective, the inventors contemplate the use of cell-free circulating tumor DNA (ctDNA) and nuclear DNA from a blood sample of a patient for the detection of microsatellite instability (MSI) in a solid tumor in the patient. Most typically, the tumor DNA is ctDNA, and the matched normal DNA is DNA from leukocytes. It is further preferred that the MSI analysis includes a step of PCR amplification of at least five MSI loci, and/or that the MSI locus is a quasi-monomorphic or monomorphic repeat marker. As before, it is contemplated that the detection of MSI includes a step of size separation chromatography without fluorescence detection and a step of comparing peak shape and position in an elution profile of a chromatogram of an amplified MSI locus.

Various objects, features, aspects and advantages of the inventive subject matter will become more apparent from the following detailed description of preferred embodiments, along with the accompanying drawing.

### BRIEF DESCRIPTION OF THE DRAWING

Prior Art FIG. 1 is a graph depicting size the distribution of amplification products for selected MSI loci using a commercially available MSI detection kit separated by capillary gel electrophoretic.

FIG. 2 is a schematic illustrating an exemplary workflow for a method according to the inventive subject matter.

FIG. 3 is one exemplary elution profile of a sample that was microsatellite stable (MSS) with tumor and normal traces.

FIG. 4 is another exemplary elution profile of a sample that was microsatellite stable (MSS) with tumor and normal traces.

FIG. 5 is a further exemplary elution profile of a sample that was microsatellite stable (MSS) with tumor and normal traces.

FIG. 6 is an exemplary elution profile of a sample with low microsatellite instability (MSI-L) with tumor and normal traces.

FIG. 7 is an exemplary elution profile of a sample with high microsatellite instability (MSI-H) with tumor and normal traces.

FIG. 8 is an exemplary elution profile of a sample with microsatellite stable (MSS) with tumor and normal traces.

FIG. 9 is an exemplary elution profile of a sample with high microsatellite instability (MSI-H) with tumor and normal traces.

FIG. 10 is an exemplary elution profile of a sample with microsatellite stable (MSS) with tumor and normal traces.

FIG. 11 is an exemplary elution profile of a sample with low microsatellite instability (MSI-L) with tumor and normal traces.

### DETAILED DESCRIPTION

The inventors have now discovered that MSI in a solid tumor can be detected without the need for a biopsy or surgery by using a blood sample from a patient. In preferred aspects, the blood sample is processed to obtain ctDNA, typically from serum, and nuclear DNA, typically from buffy coat. Most typically, ctDNA and nuclear DNA can be obtained from the same blood draw or even the same blood sample. The so obtained DNA is then employed as source material for the amplification of one or more MSI loci, and the amplicons are then subjected to size analysis, preferably without the need for fluorescent markers, which increases analytic speed and decreases cost.

As used herein, the term “tumor” refers to, and is interchangeably used with one or more cancer cells, cancer tissues, malignant tumor cells, or malignant tumor tissue, that can be placed or found in one or more anatomical locations in a human body. As used herein, the term “administering” a drug or a cancer treatment refers to both direct and indirect administration of the drug or the cancer treatment. Direct administration of the drug or the cancer treatment is typically performed by a health care professional (e.g., physician, nurse, etc.), and wherein indirect administration includes a step of providing or making available the drug or the cancer treatment to the health care professional for direct administration (e.g., via injection, oral consumption, topical application, etc.).

It should be noted that the term “patient” as used herein includes both individuals that are diagnosed with a condition (e.g., cancer) as well as individuals undergoing examination and/or testing for the purpose of detecting or identifying a condition. Thus, a patient having a tumor refers to both individuals that are diagnosed with a cancer as well as individuals that are suspected to have a cancer. As used herein, the term “provide” or “providing” refers to and includes any acts of manufacturing, generating, placing, enabling to use, transferring, or making ready to use.

Conventional MSI detection systems typically use DNA that is isolated from fresh biopsy material and a matched normal control DNA preparation from non-tumor tissue. The so obtained DNA is then used to amplify MSI loci with common MSI loci shown in Table 1. For example, two nanogram of genomic DNA was amplified and analyzed using an ABI PRISM® 3100 Genetic Analyzer with POP-4® polymer and a 36 cm capillary, and the resultant allelic patterns of the normal and test samples are shown in Prior Art FIG. 1. The presence of new alleles in the test sample (indicated by arrows) that were not present in the normal sample indicates MSI.

TABLE 1
The MSI Analysis System Locus Information.

| | | Major | Size | K562 | |
|---|---|---|---|---|---|
| Marker | GenBank ® | Repeat | Range | Alleles | Primer |
| Name | Number | Sequence | (bp)¹ | (bp) | Dye² |
| NR-21 | XM_033393 | (A)₂₁ | 94-101 | 101 | JOE |
| BAT-26 | U41210 | (A)₂₆ | 103-115 | 113 | FL |
| BAT-25 | L04143 | (A)₂₅ | 114-124 | 122 | JOE |
| NR-24 | X60152 | (A)₂₄ | 130-133 | 130 | TMR |
| MONO-27 | AC007684 | (A)₂₇ | 142-154 | 150 | JOE |
| Penta C | AL138752 | (AAAAG)₃₋₁₅ | 143-194 | 164, 174 | TMR |
| Penta D | AC000014 | (AAAAG)₂₋₁₇ | 135-201 | 168, 187 | FL |

¹Allele sizes were determined using the ABI PRISM ® 3100 Genetic Analyzer with POP-4 ® polymer and a 36 cm capillary. Rare alleles outside of these size ranges may exist. Allele sizes may vary when using different polymers or instrument configurations.
²TMR = carboxy-tetramethylrhodamine; FL = fluorescein; JOE = 6-carboxy-4′,5′-dichloro-2′,7′-dimethoxyfluorescein

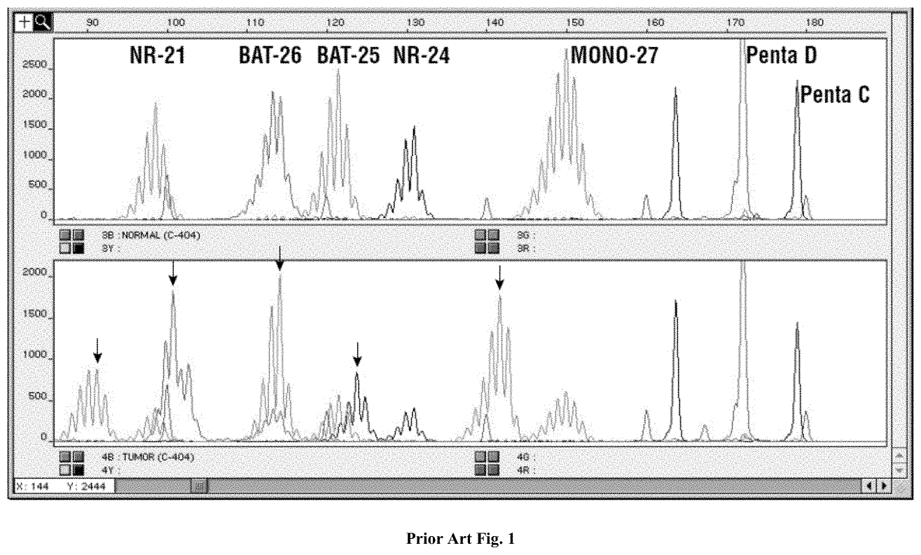

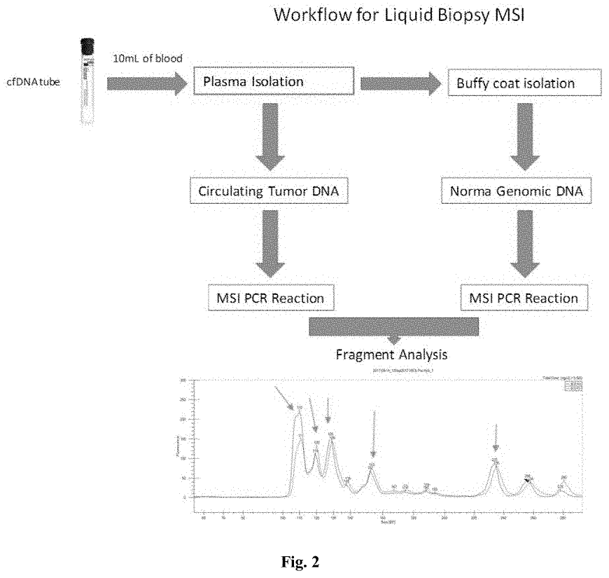

The inventors have now discovered that various materials other than a FFPE sample, a fresh tumor sample, or a tumor biopsy can be employed in a process that is simple and carries low risk to the patient as compared to a biopsy or surgery. More particularly, the inventors have discovered that any biological fluid that includes cfDNA is suitable, and especially whole blood. Advantageously, whole blood will provide in a single sample both circulating tumor DNA as well as nuclear DNA from non-tumor cells (and especially from leukocytes). Still further, it should be recognized that whole blood is also a source of cfRNA/ctRNA, which may provide further insight into the state of a tumor. FIG. 2 depicts an exemplary and schematic workflow for determination of MSI in a solid tumor from blood. Here, a blood sample is obtained from a patient, typically at a volume of between 1-50 mL, and more typically between 5-10 mL. The whole blood sample is then separated into a plasma fraction and cell-containing fraction, of which the buffy coat is used for isolation of nuclear ‘matched normal’ DNA. ctDNA is isolated form the plasma fraction, and respective PCR reactions are then performed using conventional methods on selected MSI loci to obtain amplicons that are then subjected to fragment size analysis.

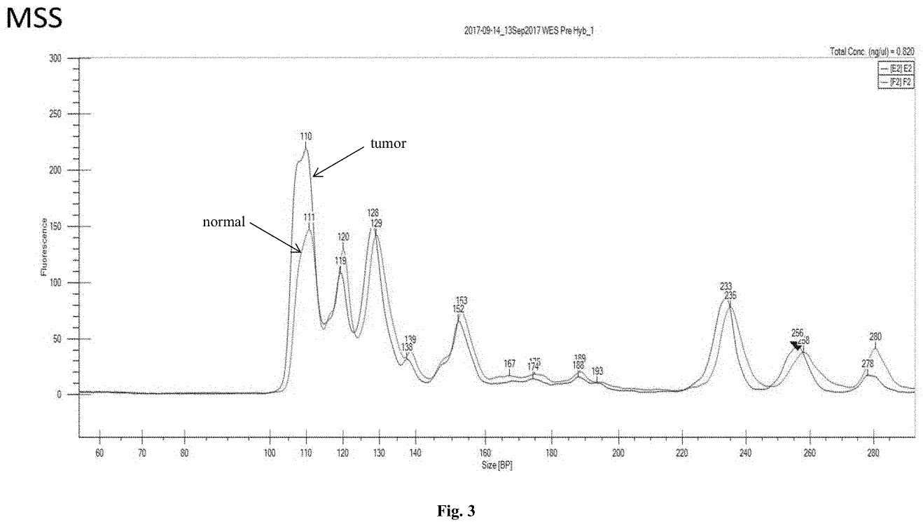

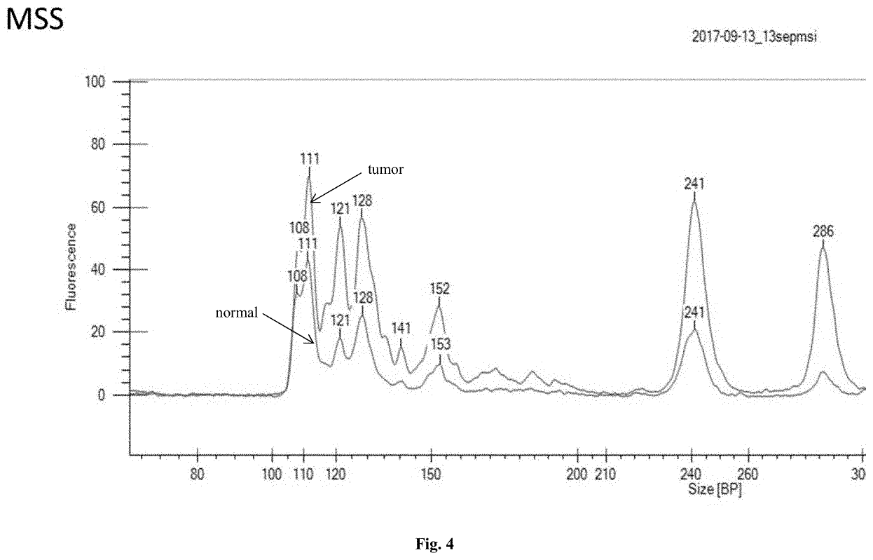

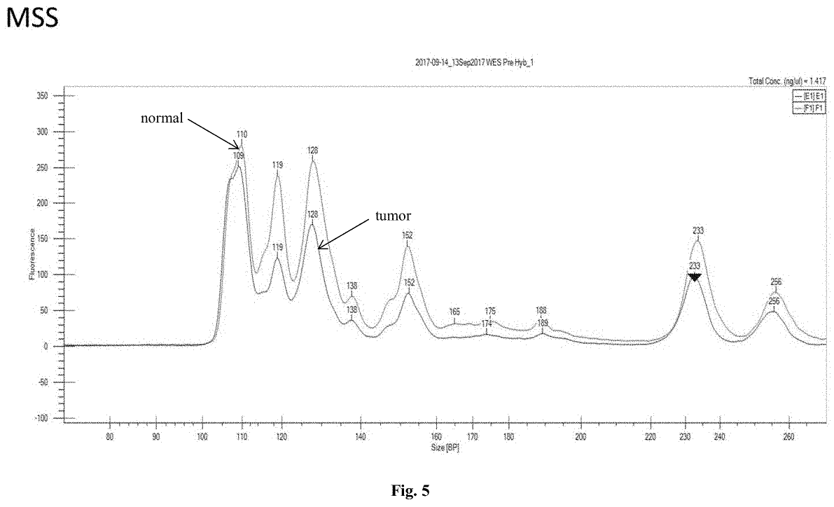

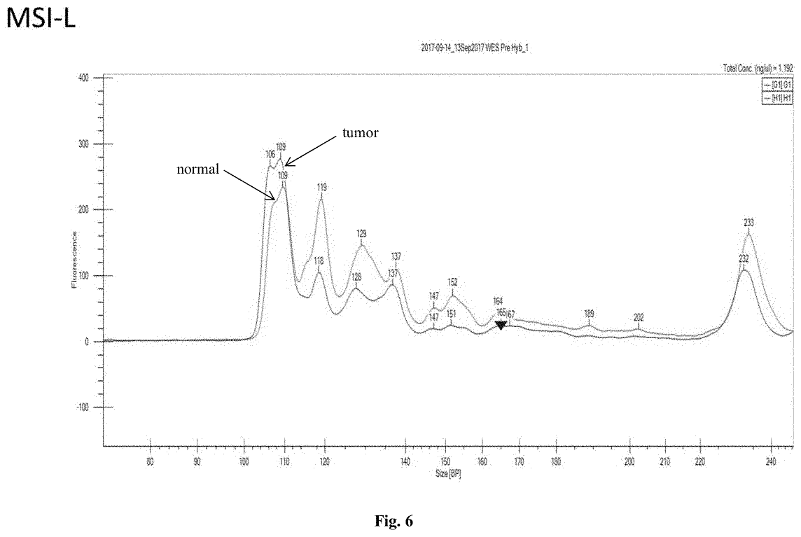

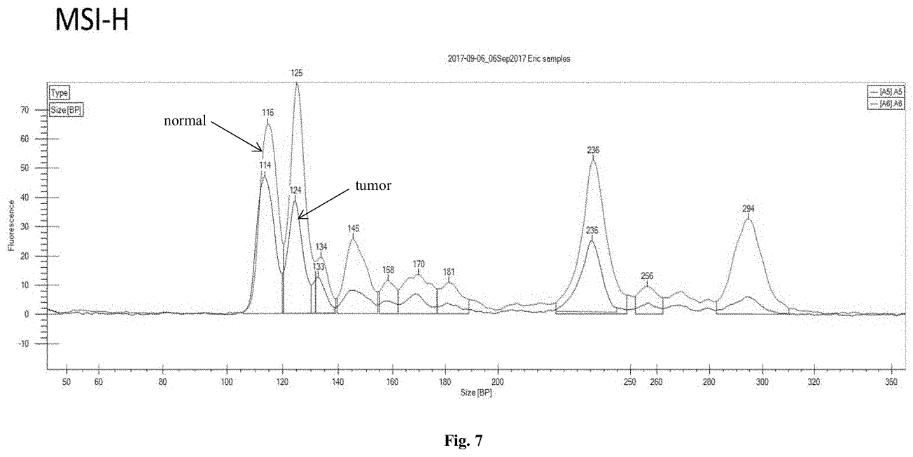

As will be readily appreciated, determination of fragment sizes and can be performed in numerous manners, and all known manners of size determination are deemed suitable for use herein. While Prior Art FIG. 1 illustrates elution profiles from fluorescently labeled amplicons, FIGS. 3-7, illustrate elution profiles from unlabeled amplicons obtained using a procedure as outlined in FIG. 2. More specifically, FIGS. 3-5 depict elution profiles from amplicons of a MSS sample where the peaks have substantially the same position as evidences by position of the maximum and where the peaks also have substantially the same shape. In contrast, FIG. 6 depicts an elution profile from amplicons of a MSI-L sample (i.e., sample with low grade MSI) where the peak shape, area-under-the-curve, and/or maximum position is altered for at least two peaks (ratio of 106 to 109 peak indicating a loss of MSI alleles at 109 and gain of alleles at 106, and ratio of 152 to 147 indicating loss of alleles at 152 and 147). FIG. 7 depicts another elution profile from amplicons of a MSI-L sample. Here, the alleles at 294 and 256 are substantially lost in the tumor, while there is an allele loss at 125 and 114, with a more pronounced loss at 125. As can be seen from a comparison of the MSS to MSI samples, overall peak shape and peak position in the elution profile of a chromatogram can be indicative of MSI as further described in more detail further below.

For example, MSI alleles can be amplified from both tumor cfDNA and nuclear DNA to produce amplicons with peak having a maximum at about 110 (bases length), 120, 128, 150, and 230. Of course, various alternative amplicons may be generated so long as such amplicons have peak maxima that can be separated or otherwise distinguished by peak shift. It should be noted that where simplified separation and detection techniques are employed, resolution of single-base differences will not be apparent as distinct peaks, however, will convolute to an overall peak profile. Such peak profile can then be analyzed to detect a shift in the position a maximum (indicative of loss of length), a shift in shape (indicative of a change in length distribution, which may be due to incomplete loss or shortening of alleles), and/or and increase or decrease of peak height relative to the matched normal sample (which may be indicative of allelic loss). As will be readily appreciated, such peak analysis can be performed by a medical professional, and more preferably by an algorithm (typically machine learning algorithm) that will then detect MSI and/or determine the type of MSI. For example, where the cfDNA and nuclear DNA samples exhibit two, three, four or more changes in peak shape, maximum, and/or ratio to another peak, MSI instability (e.g., MSI-high) is indicated. On the other hand, where the cfDNA and nuclear DNA samples exhibit one or two changes in peak shape, maximum, and/or ratio to another peak, MSI instability (e.g., MSI-low) is indicated, and where the cfDNA and nuclear DNA samples exhibit one or no changes in peak shape, maximum, and/or ratio to another peak, MSI stability (e.g., MSI-low) is indicated.

As will be readily appreciated, upon detection of MSI instability (low or high), patient will be administered one or more therapeutic agents suitable for treatment of cancer with MSI. For example, suitable agents include various checkpoint inhibitors (e.g., targeting PD-1 or CTLA4 mediated signaling), immune therapy using recombinant vaccines (e.g., viral, yeast, or bacterial), DNA damaging agents (e.g., 5-FU and/or oxaliplatin, DNA alkylating or intercalating agents, etc.) and/or agents that interfere with DNA repair (e.g., with mismatch repair, base excision repair, nucleotide excision repair, and the homology directed repair).

### Isolation and Amplification of Cell Free DNA/RNA

Any suitable methods to isolate and amplify cell free DNA/RNA are contemplated. Most typically, cell free DNA/RNA is isolated from a bodily fluid (e.g., whole blood) that is processed under a suitable conditions, including a condition that stabilizes cell free RNA. Preferably, both cell free DNA and RNA are isolated simultaneously from the same sample or draw of the patient's bodily fluid. Yet, it is also contemplated that the bodily fluid sample can be divided into two or more smaller samples from which DNA or RNA can be isolated separately. Once separated from the non-nucleic acid components, cell free DNA or RNA are then quantified, preferably using real time, quantitative PCR or real time, quantitative RT-PCR.

The bodily fluid of the patient can be obtained at any desired time point(s) depending on the purpose of the omics analysis. For example, the bodily fluid of the patient can be obtained before and/or after the patient is confirmed to have a tumor and/or periodically thereafter (e.g., every week, every month, etc.) in order to associate the cell free DNA/RNA data with the prognosis of the cancer and MSI status. In some embodiments, the bodily fluid of the patient can be obtained from a patient before and after the cancer treatment (e.g., before/after chemotherapy, radiotherapy, drug treatment, cancer immunotherapy, etc.). While it may vary depending on the type of treatments and/or the type of cancer, the bodily fluid of the patient can be obtained at least 24 hours, at least 3 days, at least 7 days after the cancer treatment. For more accurate comparison, the bodily fluid from the patient before the cancer treatment can be obtained less than 1 hour, less than 6 hours before, less than 24 hours before, less than a week before the beginning of the cancer treatment. In addition, a plurality of samples of the bodily fluid of the patient can be obtained during a period before and/or after the cancer treatment (e.g., once a day after 24 hours for 7 days, etc.).

Additionally or alternatively, the bodily fluid of a healthy individual can be obtained to compare the sequence/modification of cell free DNA, and/or quantity/subtype expression of cell free RNA. As used herein, a healthy individual refers an individual without a tumor. Preferably, the healthy individual can be chosen among group of people shares characteristics with the patient (e.g., age, gender, ethnicity, diet, living environment, family history, etc.).

Any suitable methods for isolating cell free DNA/RNA are contemplated. For example, in one exemplary method of DNA isolation, specimens were accepted as 10 ml of whole blood drawn into a test tube. Cell free DNA can be isolated from other from mono-nucleosomal and di-nucleosomal complexes using magnetic beads that can separate out cell free DNA at a size between 100-300 bps. In another example of RNA isolation, specimens were accepted as 10 ml of whole blood drawn into cell-free RNA BCT® tubes or cell-free DNA BCT® tubes containing RNA stabilizers, respectively. Advantageously, cell free RNA is stable in whole blood in the cell-free RNA BCT tubes for seven days while cell free RNA is stable in whole blood in the cell-free DNA BCT Tubes for fourteen days, allowing time for shipping of patient samples from world-wide locations without the degradation of cell free RNA. Moreover, it is generally preferred that the cell free RNA is isolated using RNA stabilization agents that will not or substantially not (e.g., equal or less than 1%, or equal or less than 0.1%, or equal or less than 0.01%, or equal or less than 0.001%) lyse blood cells. Viewed from a different perspective, the RNA stabilization reagents will not lead to a substantial increase (e.g., increase in total RNA no more than 10%, or no more than 5%, or no more than 2%, or no more than 1%) in RNA quantities in serum or plasma after the reagents are combined with blood. Likewise, these reagents will also preserve physical integrity of the cells in the blood to reduce or even eliminate release of cellular RNA found in blood cell. Such preservation may be in form of collected blood that may or may not have been separated. In less preferred aspects, contemplated reagents will stabilize cell free RNA in a collected tissue other than blood for at 2 days, more preferably at least 5 days, and most preferably at least 7 days. Of course, it should be recognized that numerous other collection modalities are also deemed appropriate, and that the cell free RNA can be at least partially purified or adsorbed to a solid phase to so increase stability prior to further processing. Similarly, cell free DNA and ctDNA can be isolated using commercially available reagents and methods, and especially preferred kits and methods include CELL-FREE DNA BCT (Streck Inc., 7002 S. 109th Street, La Vista, NE 68128).

Therefore, fractionation of plasma and extraction of cell free DNA/RNA can be done in numerous manners. In one exemplary preferred aspect, whole blood in 10 mL tubes is centrifuged to fractionate plasma at 1600 rcf for 20 minutes. The so obtained plasma is then separated and centrifuged at 16,000 rcf for 10 minutes to remove cell debris. Of course, various alternative centrifugal protocols are also deemed suitable so long as the centrifugation will not lead to substantial cell lysis (e.g., lysis of no more than 1%, or no more than 0.1%, or no more than 0.01%, or no more than 0.001% of all cells). Cell free RNA is extracted from 2 mL of plasma using Qiagen reagents. The extraction protocol was designed to remove potential contaminating blood cells, other impurities, and maintain stability of the nucleic acids during the extraction. All nucleic acids were kept in bar-coded matrix storage tubes, with DNA stored at −4° C. and RNA stored at −80° C. or reverse-transcribed to cDNA that is then stored at −4° C. Notably, so isolated cell free RNA can be frozen prior to further processing.

As will be readily appreciated, the cell-containing fraction, and especially the buffy coat (containing a large fraction of leukocytes) can be used to isolate nuclear DNA following well known protocols (see e.g., J Transl Med. 2011 Jun. 10; 9:91). Alternatively, or additionally, various commercially available kits can be used, and include QIAamp DNA Blood Mini Kit (Qiagen, 1700 Seaport Blvd, 3rd Floor, Redwood City, Calif. 94063).

MSI-Loci from ctDNA, Amplification, and Size Determination

With respect to suitable MSI loci the inventors contemplate that any known MSI locus is deemed suitable for use herein. However, particularly preferred MSI loci include mono- and di-nucleotide repeat markers, and most preferably those associated mismatch repair defects. Thus, and viewed from a different perspective, contemplated repeat markers are quasi-monomorphic (i.e., almost all individuals (e.g., at least 90%, more typically at least 95% of individuals) are homozygous for the same common allele for a given marker). Suitable loci are described in U.S. Pat. Nos. 6,150,100, 7,662,595, US2003/0113723, US 2015/0337388, and WO 2017/112738, al incorporated by reference herein. Therefore, exemplary repeats include NR-21, BAT-26, BAT-25, NR-27, NR-24, D2S123, D5S346, D175250, BAT40, MONO-27, Penta C, Penta D, D 18535, D1S2883, etc.

Depending on the particular repeat sequence/MSI locus, amplification conditions may vary as can be expected. However, the particular PCR conditions for specific MSI loci will be readily ascertainable without undue experimentation. For example, PCR conditions and reagents for amplification of NR-21, BAT26, BAT-25, NR-24, and Mono-27 is described in the product manual for the commercially available MSI Analysis System from Promega Corp. In this context, it should be appreciated that the amplification reagents may include fluorescence or otherwise labeled nucleotides, or may be performed without detectable markers. Therefore, the manner of detection will vary.

In general, for size determination of the amplicons, it is contemplated that the amplified products will be subjected to a chromatographic step that provides sufficient resolution in the size range of the amplicons. For example, suitable fragment size determination may be performed using capillary electrophoresis (e.g., using ABI PRISM 310 or Applied Biosystems 3130 Genetic Analyzer), polyacrylamide gel electrophoresis, mass spectroscopy, chip-based microfluidic electrophoresis (Methods Mol Biol. 2013; 919:287-96), and denaturing high performance liquid chromatography. In general, it is contemplated that size determination is performed in parallel with the patient matched normal sample to detect a shift in allelic size distribution. Such size determination and methods are well known in the art and do not require undue experimentation.

Further improvements can be added to contemplated systems and methods by use of non-fluorescent amplicons. As can be seen from Prior Art FIG. 1, the fluorescence signals for each amplicon size are resolved at a single base pair level and produce independent signals. However, such resolution is typically lost when other detection methods are employed, such as UV detection, amperometric detection, etc. Nevertheless, when non-fluorescence methods are used, individual peaks will superimpose to a final signal, which can be mathematically stated as shown in equations I and II

f(x)=[f1(x),f2(x), . . . ,fn(x)] Eq.I

The final scale signal can be expressed as a superposition of independent signals:

ff=[ff1,ff2, . . . ,ffn] Eq.II

g(x)=ff·f(x)

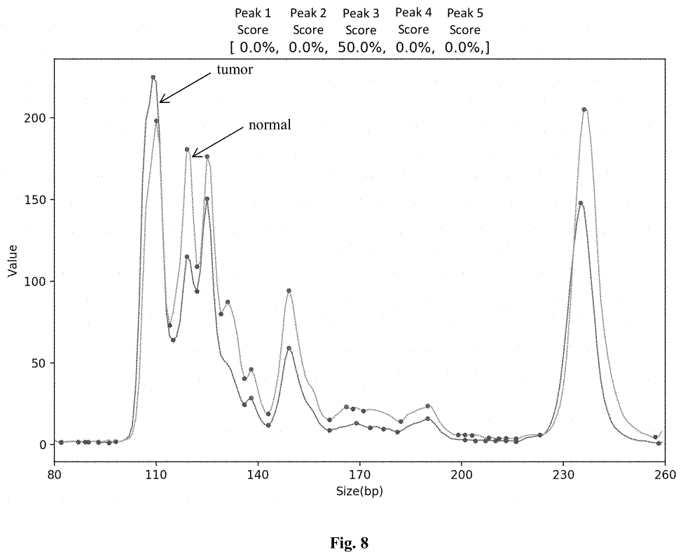

The final signal (calculated value according to above equations) of each individual peak will be compared against cut-off value 30%. If that value is >=30% the individual peak will be determined as a shift. Two or more peak shifts will determine sample's status as MSI High (see FIGS. 8-11).

The inventors now contemplate that independent signals can be recovered with independent component analysis, provided sufficient training data are available. While linear superposition and other methods may be employed, Neural Networks may be a more advantageous choice as the signal can be converted to fixed size features.

For example, in a rule based system, the following procedures may be used: (a) Find all maximum and minimum for both reference and testing sample; (b) ensure the quality of peak; (c) Find the primary peaks for reference sample; (d) Find the primary peaks based on reference peak for testing sample; (e) compute information associated with each primary peaks such as peak value ratio (prev, curr), (curr, next), peak area ratio, and peak width; (f) Compute the probability for each testing primary peak; and (g) Evaluate MSI-H, MSI-L, or MSS possibility based on peak probability. Such approach will typically not require training samples and may be used to improve understanding of data.

In another, more preferred example, a hand crafted feature based system may be employed, where the feature vector includes the following quantity for each primary peak: peak value, peak width, peak area, peak value ratio between (prev, curr), (curr, next), and peak area ratio between (prev, curr), (curr, next). Thus, the feature vector size in this example will be 5*5=25 for 5 primary peaks. Classification can be performed using Supporting Vector Method or Random Forest. Most commonly, training sample size will be in excess of 100 samples.

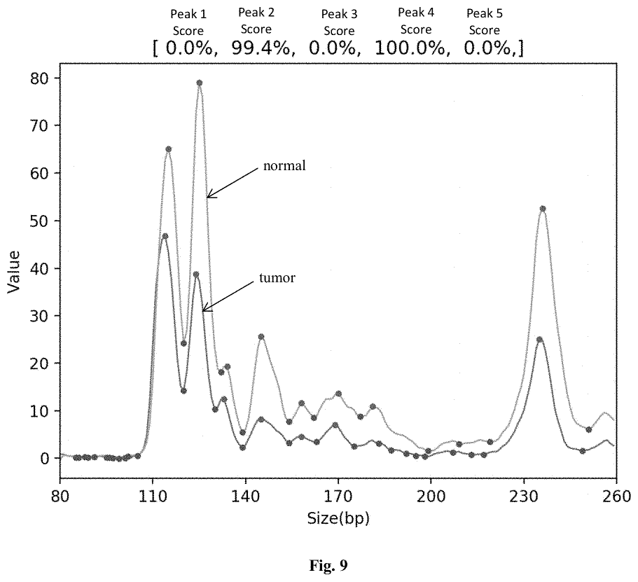

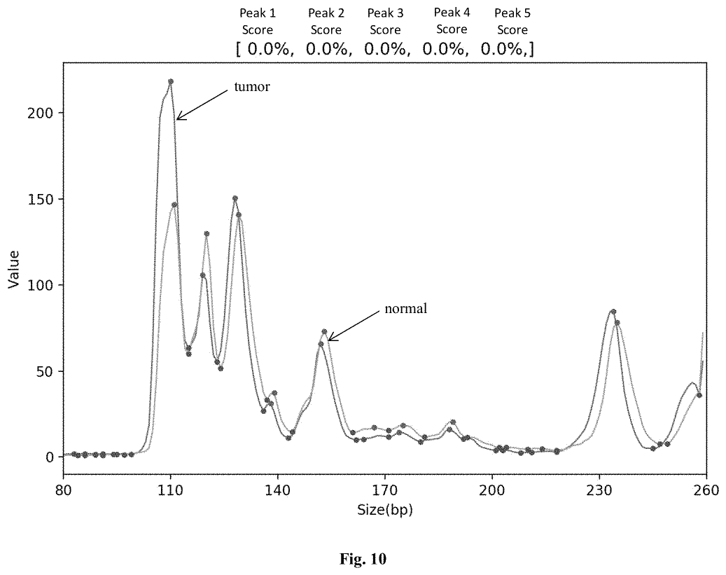

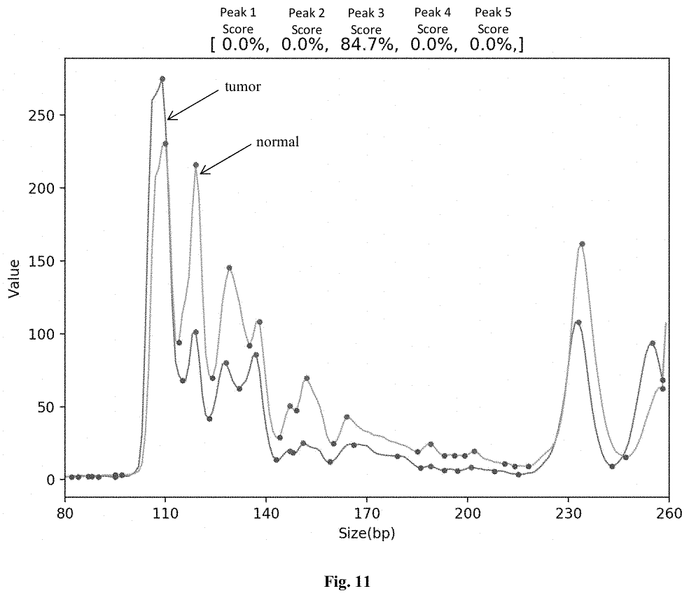

Alternatively, a raw feature based system may be employed. For example, the feature vector could include all fluorescence reading, with cubic spline interpolation so that the data is evenly distributed in base pair size (between [80, 250], feature size is 170). Siamese Neural Networks could be used with the reference sample as one fully connected network, and the test sample as second fully connected network. Most commonly, training sample size will be in excess of 1,000 samples. FIGS. 8-11 illustrate elution profiles for amplicons without use of fluorescence markers (e.g., microfluidic based on-chip electrophoresis on the Agilent 2100 Bioanalyzer). Here, blood samples from patients with solid tumors (colorectal cancer) and known tumor MSI status (previously established from fresh tumor sample using conventional methods) were obtained. cfDNA and nuclear DNA were prepared as noted above and selected MSI alleles amplified following known methods. Image analysis as described above was implemented and salient peak features detected in the elution profiles are exemplarily illustrated in FIGS. 8-11. More particularly, FIGS. 8 and 10 illustrate examples of microsatellite stable (MSS). FIG. 9 illustrates one example of high microsatellite instability (MSI-H) but FIG. 11 illustrates one example of low microsatellite instability (MSI-L).

Other Sequences of Interest for cfRNA Analysis

The inventors further contemplate that tumor cells and/or some immune cells interacting or surrounding the tumor cells release cell free DNA/RNA to the patient's bodily fluid, and thus may increase the quantity of the specific cell free DNA/RNA in the patient's bodily fluid as compared to a healthy individual. As noted above, the patient's bodily fluid includes, but is not limited to, blood, serum, plasma, mucus, cerebrospinal fluid, ascites fluid, saliva, and urine of the patient. Alternatively, it should be noted that various other bodily fluids are also deemed appropriate so long as cell free DNA/RNA is present in such fluids. The patient's bodily fluid may be fresh or preserved/frozen. Appropriate fluids include saliva, ascites fluid, spinal fluid, urine, etc., which may be fresh or preserved/frozen.

The cell free RNA may include any types of DNA/RNA that are circulating in the bodily fluid of a person without being enclosed in a cell body or a nucleus. Most typically, the source of the cell free DNA/RNA is the tumor cells. However, it is also contemplated that the source of the cell free DNA/RNA is an immune cell (e.g., NK cells, T cells, macrophages, etc.). Thus, the cell free DNA/RNA can be circulating tumor DNA/RNA (ctDNA/RNA) and/or circulating free DNA/RNA (cf DNA/RNA, circulating nucleic acids that do not derive from a tumor). While not wishing to be bound by a particular theory, it is contemplated that release of cell free DNA/RNA originating from a tumor cell can be increased when the tumor cell interacts with an immune cell or when the tumor cells undergo cell death (e.g., necrosis, apoptosis, autophagy, etc.). Thus, in some embodiments, the cell free DNA/RNA may be enclosed in a vesicular structure (e.g., via exosomal release of cytoplasmic substances) so that it can be protected from nuclease (e.g., RNAase) activity in some type of bodily fluid. Yet, it is also contemplated that in other aspects, the cell free DNA/RNA is a naked DNA/RNA without being enclosed in any membranous structure, but may be in a stable form by itself or be stabilized via interaction with one or more non-nucleotide molecules (e.g., any RNA binding proteins, etc.).

In view of the above, it is contemplated that the cell free DNA/RNA can be any type of DNA/RNA which can be released from either cancer cells or immune cell. Thus, the cell free DNA may include any whole or fragmented genomic DNA, or mitochondrial DNA, and the cell free RNA may include mRNA, tRNA, microRNA, small interfering RNA, long non-coding RNA (lncRNA). Most typically, the cell free DNA is a fragmented DNA typically with a length of at least 50 base pair (bp), 100 base pair (bp), 200 bp, 500 bp, or 1 kbp. Also, it is contemplated that the cell free RNA is a full length or a fragment of mRNA (e.g., at least 70% of full-length, at least 50% of full length, at least 30% of full length, etc.). While cell free DNA/RNA may include any type of DNA/RNA encoding any cellular, extracellular proteins or non-protein elements, it is preferred that at least some of cell free DNA/RNA encodes one or more cancer-related proteins, or inflammation-related proteins. In another example, alternative or additionally contemplated cfDNAs/mRNAs are fragments of or those encoding a full length or a fragment of inflammation-related proteins, including, but not limited to, HMGB1, HMGB2, HMGB3, MUC1, VWF, MMP, CRP, PBEF1, TNF-α, TGF-0, PDGFA, IL-1, IL-2, IL-3, IL-4, IL-5, IL-6, IL-7, IL-8, IL-9, IL-10, IL-12, IL-13, IL-15, IL-17, Eotaxin, FGF, G-CSF, GM-CSF, IFN-γ, IP-10, MCP-1, PDGF, and hTERT, and in yet another example, the cell free mRNA encoded a full length or a fragment of HMGB1. In still another example, cell free DNAs/mRNAs are fragments of or those encoding a full length or a fragment of DNA repair-related proteins or RNA repair-related proteins, such as Base excision repair (BER) related genes, Mismatch repair (MMR) related genes, Nucleotide excision repair (NER) related genes, Homologous recombination (HR), and Non-homologous end-joining (NHEJ) related genes.

Where desired, cell free DNAs/mRNAs are fragments of or those encoding a full length or a fragment of a gene not associated with a disease (e.g., housekeeping genes), including those related to transcription factors (e.g., ATF1, ATF2, ATF4, ATF6, ATF7, ATFIP, BTF3, E2F4, ERH, HMGB1, ILF2, IER2, JUND, TCEB2, etc.), repressors (e.g., PUF60), RNA splicing (e.g., BAT1, HNRPD, HNRPK, PABPN1, SRSF3, etc.), translation factors (EIF1, EIF1AD, EIF1B, EIF2A, EIF2AK1, EIF2AK3, EIF2AK4, EIF2B2, EIF2B3, EIF2B4, EIF2S2, EIF3A, etc.), tRNA synthetases (e.g., AARS, CARS, DARS, FARS, GARS, HARS, IARS, KARS, MARS, etc.), RNA binding protein (e.g., ELAVL1, etc.), ribosomal proteins (e.g., RPL5, RPL8, RPL9, RPL10, RPL11, RPL14, RPL25, etc.), mitochondrial ribosomal proteins (e.g., MRPL9, MRPL1, MRPL10, MRPL11, MRPL12, MRPL13, MRPL14, etc.), RNA polymerase (e.g., POLR1C, POLR1D, POLR1E, POLR2A, POLR2B, POLR2C, POLR2D, POLR3C, etc.), protein processing (e.g., PPID, PPI3, PPIF, CANX, CAPN1, NACA, PFDN2, SNX2, SS41, SUMO1, etc.), heat shock proteins (e.g., HSPA4, HSPA5, HSBP1, etc.), histone (e.g., HIST1HSBC, H1FX, etc.), cell cycle (e.g., ARHGAP35, RAB10, RAB11A, CCNY, CCNL, PPP1CA, RAD1, RAD17, etc.), carbohydrate metabolism (e.g., ALDOA, GSK3A, PGK1, PGAM5, etc.), lipid metabolism (e.g., HADHA), citric acid cycle (e.g., SDHA, SDHB, etc.), amino acid metabolism (e.g., COMT, etc.), NADH dehydrogenase (e.g., NDUFA2, etc.), cytochrome c oxidase (e.g., COX5B, COX8, COX11, etc.), ATPase (e.g. ATP2C1, ATP5F1, etc.), lysosome (e.g., CTSD, CSTB, LAMP1, etc.), proteasome (e.g., PSMA1, UBA1, etc.), cytoskeletal proteins (e.g., ANXA6, ARPC2, etc.), and organelle synthesis (e.g., BLOC1S1, AP2A1, etc.).

It should be apparent to those skilled in the art that many more modifications besides those already described are possible without departing from the inventive concepts herein. The inventive subject matter, therefore, is not to be restricted except in the scope of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise. Where the specification claims refers to at least one of something selected from the group consisting of A, B, C . . . and N, the text should be interpreted as requiring only one element from the group, not A plus N, or B plus N, etc.

1. A method of detecting microsatellite instability (MSI) in a solid tumor, the method comprising:
isolating a cell-contain ng fraction and a cell-depleted fraction from a blood sample of a patient having the solid tumor;
isolating cell-free circulating tumor DNA (ctDNA) from the cell-depleted fraction;
isolating nuclear DNA from the cell-containing fraction; amplifying at least one MSI locus in the ctDNA and in the nuclear DNA; and
detecting a size difference between the amplified MSI locus in the ctDNA and the amplified MSI locus in the nuclear DNA.

2. The method of claim 1, wherein the cell-containing fraction is a buffy coat fraction.

3. The method of any claim 1, wherein the step of amplifying at least one MSI locus comprises amplifying at least three MSI loci.

4. The method of any claim 3, wherein the step of amplifying at least one MSI locus comprises amplifying at least five MSI loci.

5. The method of any claim 1, wherein the at least one MSI locus is a quasi-monomorphic or monomorphic repeat marker.

6. The method of any claim 1, wherein the at least one MSI locus includes a mononucleotide repeat or a dinucleotide repeat.

7. The method of any claim 1, wherein the at least one MSI locus is selected from the group consisting of NR-21, BAT-26 BAT-25 NR-24, and MONO-27.

8. The method of any claim 1, wherein the step of detecting the size difference is performed using capillary electrophoresis, polyacrylamide gel electrophoresis, mass spectroscopy, chip-based microfluidic electrophoresis, and denaturing high performance liquid chromatography.

9. The method of any claim 1, wherein the step of detecting the size difference comprises a step of comparing peak shape and position in an elution profile of a chromatogram of the amplified MSI locus.

10. The method of claim 9, wherein the peak shape is area under the curve and/or peak height.

11. The method of claim 9, wherein the step of comparing comprises a step of independent component analysis.

12. A method of detecting microsatellite instability (MSI) in a solid tumor, the method comprising:
obtaining tumor and matched normal DNA from a blood sample of a patient having the solid tumor; using the tumor and matched normal DNA from the blood sample as source material for MSI analysis.

13. The method of claim 12, wherein the tumor DNA is ctDNA, and wherein the matched normal DNA is DNA from leukocytes.

14. The method of claim 12, wherein the MSI analysis includes a step of PCR amplification of at least one MSI locus.

15. The method of claim 12, wherein the MSI analysis includes a step of capillary electrophoresis and fluorescence detection.

16. The method of claim 12, wherein the MSI analysis includes a step of size separation chromatography without fluorescence detection.

16-20. (canceled)
